(19) United States
(12) Patent Application Publication
(10) Pub. No.: US 2013/0149192 A1
(43) Pub. Date: Jun. 13, 2013

(54) METHOD AND STRUCTURE FOR GENERATING AND RECEIVING ACOUSTIC SIGNALS AND ERADICATING VIRAL INFECTIONS

(71) Applicant: John P. Keady, Fairfax Station, VA (US)

(72) Inventor: John P. Keady, Fairfax Station, VA (US)

(21) Appl. No.: 13/609,208

(22) Filed: Sep. 10, 2012

Related U.S. Application Data
(60) Provisional application No. 61/532,099, filed on Sep. 8, 2011.

(51) Int. Cl. A61L 2/02

(52) U.S. Cl. CPC A61L 2/02
USPC 422/20; 422/127

(57) ABSTRACT

At least embodiment is directed to a method of viral eradication which includes: delivering an acoustic wave to a virally infected region; tuning a frequency of the acoustic wave to a resonance frequency of a target virus in the virally infected region; and applying the acoustic wave to the virally infected region for a period of time necessary to eradicate at least 25% of the target virus per cubic mm of the virally infected region.

### CROSS-REFERENCE TO RELATED APPLICATIONS

This application claims the benefit of U.S. provisional patent application No. 61/532,099 filed 8 Sep. 2011. The disclosure of which is incorporated herein by reference in its entirety.

### FIELD OF THE INVENTION

The present invention relates to devices that can be used to generate of receive acoustical energy and more particularly, though not exclusively, a a device that uses acoustical energy to destroy viruses.

### BACKGROUND OF THE INVENTION

Viruses include a genome and often enzymes encapsulated by protein capsid, with often a lipid envelope. A virus must subjugate a host to reproduce, and various methods are used to attack viruses throughout their life cycle. Two common methods used are vaccines and anti-viral drugs. Vaccines can be effective on stable viruses but not on infected patients or fast mutating viruses. Anti-viral drugs target viral proteins. The disadvantage of anti-viral drugs is the eventual pathogen mutation over time and the hazard of side effects if the viral proteins are similar to human proteins.

The market for anti-viral drugs totals in the billions of dollars. Generics in global antivirals market are estimated to be $4.2 billion in 2010 and are forecast to reach $9.2 billion by 2018. Generics in the HIV market accounted for 46% of market share in total generic antivirals market in 2010, while generic herpes therapeutics accounted for 39.6% of market share. Generic influenza therapeutics accounted for 1% of total market share.

It has been reported that in 2002, the annual treatment for HIV/AIDS cost an average of $9,971. This grew substantially at a compound average growth rate (CAGR) of 3.2% to $12,829 in 2010. Deaths in 2011 as a result from HIV/AIDS was greater than 1 million worldwide.

A method of permanent viral eradication, without drugs, without the possibility of pathogen mutation, and with equipment that can treat patients in few visits, would save millions of lives and billions of dollars each year. Additionally the technique could be applied to sterilizing medical instruments.

### BRIEF DESCRIPTION OF THE DRAWINGS

Exemplary embodiments of present invention will become more fully understood from the detailed description and the accompanying drawings, wherein:

FIG. 1 illustrates general physiology of a wave (e.g., acoustic, ElectroMagnetic (EM), phonon) impinging upon a simple model of a virus;

FIG. 2 illustrates a simplified model of an acoustic wave penetrating various levels of materials (e.g., skin, vascular walls, blood), where the transmitted acoustic waves set up resonances in the viruses that eventually ruptures the virus (e.g., exceeds the elastic limit);

FIG. 3 illustrates at least one exemplary embodiment of generating acoustic waves using an oscillating fluid medium (e.g., a magneto responsive fluid (MR));

FIG. 4 illustrates at least one exemplary embodiment of generating acoustic waves using an oscillating fluid medium (e.g., an electro responsive fluid (ER));

FIG. 5 illustrates at least one exemplary embodiment of generating acoustic waves using photo acoustics;

FIG. 6 illustrates at least one exemplary embodiment of generating acoustic waves using photo acoustics in one medium and coupling the generated acoustic waves into a second medium;

FIG. 7 illustrates at least one exemplary embodiment of generating acoustic waves, with optional cooling coils and optional EM shielding;

FIG. 8 illustrates data from a prototype generating an acoustic wave in from an oscillating MR fluid bladder by changing the magnetic field at low frequencies;

FIG. 9 illustrates an example of using an acoustic generated acoustic wave to sterilize a viral infected medium in a test tube;

FIG. 10 illustrates at least one exemplary embodiment of coupling an acoustic wave (e.g. via acoustic waveguide) to a ring device on a finger to input the acoustic waves into the patient body;

FIG. 11 illustrates at least one exemplary embodiment of coupling an acoustic wave (e.g. via acoustic waveguide) to an instrument (e.g., medical surgical instrument);

FIG. 12 illustrates at least one exemplary embodiment of coupling an acoustic wave (e.g. via acoustic waveguide) to a limb wrap device, that can be wrapped around a limb to input the acoustic waves into the patient body; and

FIG. 13 illustrates at least one exemplary embodiment of coupling an acoustic wave (e.g. via acoustic waveguide) to a wrist wrap device, that can be wrapped around a wrist to input the acoustic waves into the patient body.

### DETAILED DESCRIPTION OF EMBODIMENTS

The following description of exemplary embodiment(s) is merely illustrative in nature and is in no way intended to limit the invention, its application, or uses.

Exemplary embodiments are directed to a device to generate or receive acoustic waves, that can be used as an acoustic source (e.g., speaker) and acoustic microphone (e.g., microphone). In particular exemplary embodiments discussed utilize fluid-based or laser-based generated acoustic waves to generate high frequency acoustic waves to generate acoustic resonance to deactivate/destroy viruses (MHz to GHz). Note that similar exemplary embodiments can generate hearing acoustic and ultrasonic frequencies (e.g., 10 Hz-50 kHz) and can be used as speakers and microphones.

At least one exemplary embodiment is directed to generating a high frequency acoustic source to set up acoustic resonance in live viruses.

Processes, techniques, apparatus, and materials as known by one of ordinary skill in the art may not be discussed in detail but are intended to be part of the enabling description where appropriate. For example specific materials may not be listed for achieving each of the targeted properties discussed, however one of ordinary skill would be able, without undo experimentation, to determine the materials needed given the enabling disclosure herein. Additionally various techniques, formulas, in acoustical physics and photoacoustics is assumed. Thus the contents of “Photoacoustic Imaging and Spectroscopy” edited by Lihong V. Wang, CRC Press, Optical Science and Engineering #144 is incorporated by reference in its entirety, as is the “fundamentals of physical acoustics” by David T. Blackstock, ISBN 0-471-31979-1 which is also incorporated by reference in its entirety.

Notice that similar reference numerals and letters refer to similar items in the following figures, and thus once an item is defined in one figure, it may not be discussed or further defined in the following figures. Processes, techniques, apparatus, and materials as known by one of ordinary skill in the relevant art may not be discussed in detail but are intended to be part of the enabling description where appropriate.

FerroFluids (FF) and Magnetorheological Fluids (MRF): Ferrofluids (also referred to as magnetoresponsive fluids (MR)) can be composed of nanoscale particles (diameter usually 10 nanometers or less) of magnetite, hematite or some other compound containing iron. This is small enough for thermal agitation to disperse them evenly within a carrier fluid, and for them to contribute to the overall magnetic response of the fluid. Ferrofluids can include tiny iron particles covered with a liquid coating, also surfactant that are then added to water or oil, which gives them their liquid properties.

Ferrofluids are colloidal suspensions—materials with properties of more than one state of matter. In this case, the two states of matter are the solid metal and liquid it is in this ability to change phases with the application of a magnetic field allows them to be used as seals, lubricants, and may open up further applications in future nanoelectromechanical systems. In at least one embodiment a sample of ferrofluid can be mixed with various other fluids (e.g., water, mineral oil, alcohol) to acquire various desired properties. For example when mixed with water and a magnetic field is applied the ferrofluid will separate from the water pushing the water in the opposite direction from the ferrofluid. Such a system can be used as a pump to move fluid from one side of a bladder to another, or even into a separate region, for example where the water can react to an agent when the ferrofluid would not. Another example of a benefit to mixing is to vary the viscosity of the fluid. If the ferrofluid is mixed with mineral oil, the net fluid is less viscous and more easily moved, while remaining mixed when a magnetic field is applied. If the net fluid is in a reservoir chamber one can move the fluid into a different chamber by application of a magnetic field. Note that the discussion above applies equally well for an ER fluid where electric fields are applied instead of magnetic fields.

True ferrofluids are stable. This means that the solid particles do not agglomerate or phase separate even in extremely strong magnetic fields. However, the surfactant tends to break down over time (a few years), and eventually the nano-particles will agglomerate, and they will separate out and no longer contribute to the fluid's magnetic response. The term magnetorheological fluid (MRF) refers to liquids similar to ferrofluids (FF) that solidify in the presence of a magnetic field. Magnetorheological fluids have micrometer scale magnetic particles that are one to three orders of magnitude larger than those of ferrofluids. The specific temperature required varies depending on the specific compounds used for the nano-particles.

The surfactants used to coat the nanoparticles include, but are not limited to: oleic acid; tetramethylammonium hydroxide; citric acid; soy lecithin These surfactants prevent the nanoparticles from clumping together, ensuring that the particles do not form aggregates that become too heavy to be held in suspension by Brownian motion. The magnetic particles in an ideal ferrofluid do not settle out, even when exposed to a strong magnetic, or gravitational field. Steric repulsion then prevents agglomeration of the particles. While surfactants are useful in prolonging the settling rate in ferrofluids, they also prove detrimental to the fluid's magnetic properties (specifically, the fluid's magnetic saturation). The addition of surfactants (or any other foreign particles) decreases the packing density of the ferroparticles while in its activated state, thus decreasing the fluids on-state viscosity, resulting in a “softer” activated fluid. While the on-state viscosity (the “hardness” of the activated fluid) is less of a concern for some ferrofluid applications, it is a primary fluid property for the majority of their commercial and industrial applications and therefore a compromise must be met when considering on-state viscosity versus the settling rate of a ferrofluid.

Ferrofluids in general comprise a colloidal suspension of very finely-divided magnetic particles dispersed in a liquid carrier, such as water or other organic liquids to include, but not limited to: liquid hydrocarbons, fluorocarbons, silicones, organic esters and diesters, and other stable inert liquids of the desired properties and viscosities. Ferrofluids of the type prepared and described in U.S. Pat. No. 3,917,538, issued Nov. 4, 1975, hereby incorporated by reference in its entirety, may be employed. The ferrofluid is selected to have a desired viscous-dampening viscosity in the field; for example, viscosities at 25.degree. C. of 100 to 5000 cps at 50 to 1000 gauss saturation magnetization of the ferrofluid such as a liquid ferrofluid having a viscosity of about 500 to 1500 cps and a magnetic saturation of 200 to 600 gauss. The magnetic material employed may be magnetic material made from materials of the Alnico group, rare earth cobalt, or other materials providing a magnetic field, but typically comprises permanent magnetic material. Where the permanent magnetic material is used as the seismic mass, it is axially polarized in the housing made of nonferromagnetic material, such as aluminum, zinc, plastic, etc., and the magnet creates a magnetic-force field which equally distributes the enclosed ferrofluid in the annular volume of the housing and on the planar faces of the housing walls.

The proposed method utilizes a physical principle well known in the physical sciences called resonance. When an engineering object is designed and built, resonance must be taken into account to avoid catastrophic build up of vibrations that occur at the resonant frequency of the object. The proposed method would gradually build up vibration energy in a virus by impacting the virus with acoustic waves at the virus's resonant frequency, which is a function of the size, density and geometry of the virus. The method, applied for a period of time, would tear apart the targeted virus in a patient's body without interjecting any anti-viral chemicals into the patient's system. The remaining portions of the virus could be used by the immune system of the patient to develop antibodies.

The proposed technique is resonance based and chemical free, and therefore not adaptable by a virus. Resonance is a design concern for any mechanical design. Forced vibrations at the natural frequency of a system can result in resonance buildup to levels that destroy the system, even if the amplitude of the forced vibration is relatively low. The natural frequency of a system depends on several factors, such as size, geometric configuration, density, and damping of the suspension medium.

An acoustical wave impinging upon the object at the resonance frequency will result in a gradual internal amplitude increase to the point in which the object tears itself apart (e.g., exceeds its elastic strength). If a virus is the object, such resonance will be able to tear apart any virus provided the acoustic wave is not detrimentally damped in the medium (e.g. blood) in which the virus lies. The technique can additionally be used for instrument (e.g., medical instrument) sterilization. Different virus's will have different resonant frequencies, and those frequencies will be different than neighboring cells and cellular structure (e.g., size and density differences) such that the virus will be able to be targeted directly without damaging healthy cells.

In general a virus can range in diameter from 20 nm to about 300 nm. If the resonant frequency is solely based upon viral size the needed acoustic frequency would be in the GHz range. The actual viral resonant frequencies are unknown. A simplified air bubble in water model provides a resonant frequency of about 65.6 MHz for a dimension of about 100 nm, much smaller than reported by molecular models.

FIG. 1 below provides a simplified view of an acoustic wave 100 passing through a virus 110. Using a simplified assumption (equations (1) and (2) that the speed of sound in the virus is identical to the medium in which it resides, one can see that if the wavelength 140 (λ) of the impinging acoustic wave is twice the viral dimension 170 (d), a new impinging wavelength will strike the virus (e.g., wave part WA 120 impinges virus at location VA 150) when the internal viral acoustic wave 180 reaches the same location (VA 150) after internal reflection (e.g., internal wave reflects from VB 160 back to VA 150 when WB 130 reaches VA 150), allowing resonance buildup. If we assume that the medium is water with a speed of sound of 1500 m/sec, and a viral dimension 170 (d) of 100 nm, we obtain a value of 7.5 GHz. If we reduce the viral dimension to say 10 nm the resonance frequency using this simplified model would be 75 GHz. This matches well more complicated molecular models.

d=λ/2 (1)

f=(1500 m/s)/(2*100 nm)=7.5 GHz (2)

A more detailed analysis is provided by equations (3)-(5). Resonance can occur when the time of travel of the internal acoustic wave of the virus from VA->VB->VA matches the period or time of travel of a single wavelength of the ambient acoustic wave. The time of travel (tv) of the internal viral acoustic wave 180 is given in equation (3) and it is a function of the dimension 170 of the virus (d) and the speed of sound in the virus (Cv).

tv=2d/Cv (3)

The period (T) of the ambient acoustic wave 100 is an inverse of the frequency (f) of the wave (in Hz) as expressed by equation (4), and the frequency can be related to the speed of sound in the ambient medium (C0) and the wavelength (λ) of the wave.

T=1/f=λ/C0 (4)

To acquire the condition necessary for resonance to occur, we can set equal T and tv to obtain the expression in equation (5).

T=tv=2d/Cv=λ/C0=1/f (5)

For the simplified case discussed above where Co=Cv, equation (5) reduces to equation (1).

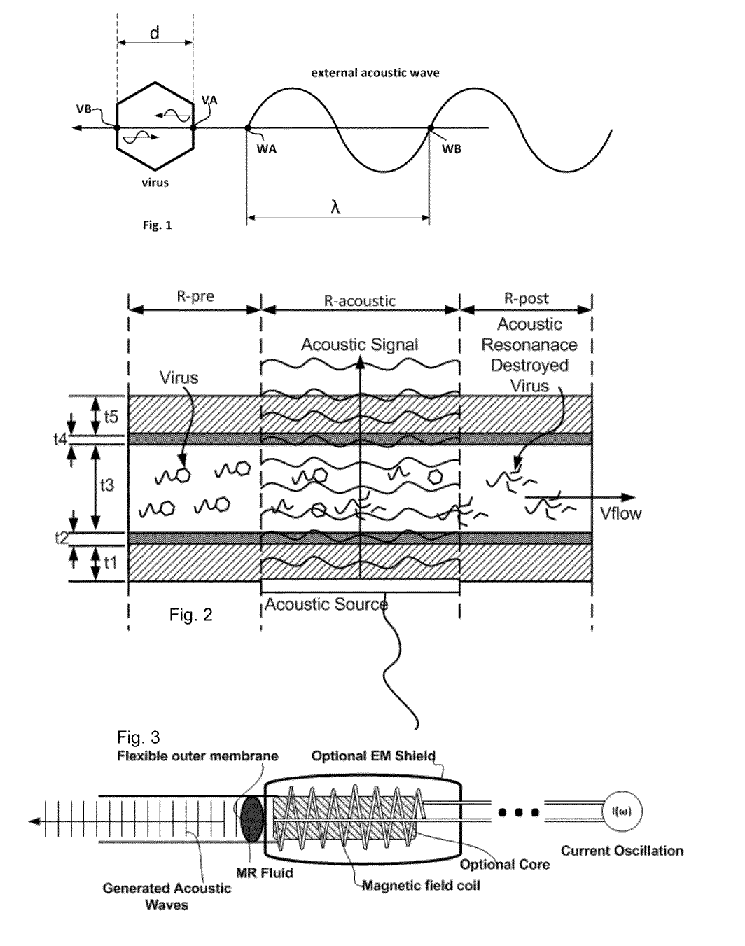

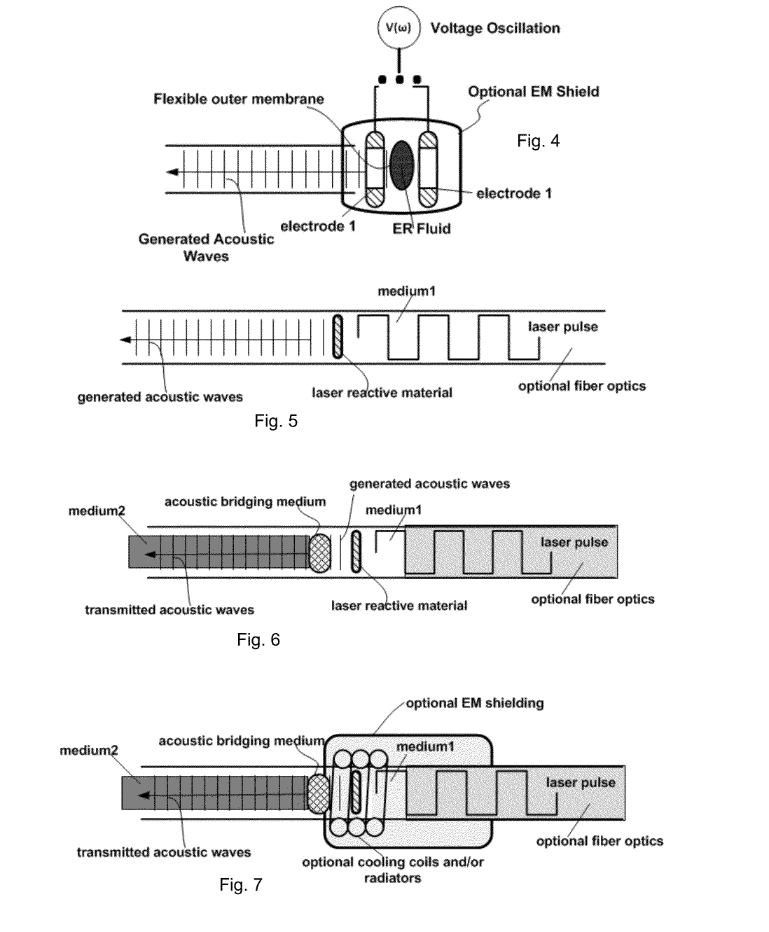

FIG. 2 illustrates a simplified model of an acoustic wave penetrating various levels of materials (e.g., skin, vascular walls, blood), where the transmitted acoustic waves set up resonances in the viruses that eventually ruptures the virus (e.g., exceeds the elastic limit). In the simplified view an acoustic source 200 (e.g., as described in FIG. 3, FIG. 4, FIG. 5, FIG. 6, and FIG. 7) generates an acoustic wave to a virally infected region for a period of time necessary to eradicate at least a target % (e.g., 5%, 10%, 25%, 50%, 75%) of the target virus per cubic mm of the virally infected region. The virally affected region is the region in which the acoustic wave travels that contains the target viruses. For example acoustic source 200 generates the acoustic signal 240, which passes through a portion of a region that contains the target virus 220. The region through which the acoustic signal passes can include multiple levels 250, 260, 270, 280, and 290 (e.g., skin tissue, vascular walls, blood). Each level can affect the acoustic energy (e.g., damping, dispersion) of the acoustic signal. The acoustic signal can be any type of periodic wave (e.g. sine wave, comb functions, ramp functions, cosine waves) that can be used to set up resonance frequencies in the targeted viruses. Since each target virus has a unique size, and composition each virus will have a unique resonance frequency. The acoustic wave 200 can be tuned to the viral resonant frequency to eradicate just the target virus.

FIG. 3 illustrates at least one exemplary embodiment 300 of generating acoustic waves using an oscillating fluid medium (e.g., a magneto responsive fluid (MR) or any other field responsive fluid). For example a field responsive medium 320 (e.g., MR and/or ER Fluid) enclosed with at least one side having a flexible membrane 310 (e.g., silicone, urethane, rubber) can be oscillated in response to an oscillating field (e.g., magnetic and/or electric field). In the non-limiting example illustrated in FIG. 3, the field responsive medium 320 oscillates generating an acoustic wave 380, which can be guided by a waveguide 390. The optional waveguide 380 can be chosen so that the propagation modes are at the desired frequencies. A field oscillating device (e.g., magnetic coil 370 and optionally core 360 (e.g., ferrous)) is configured to generate an oscillating field at a target frequency moving the field responsive medium 320 at the target frequency, where movement of the field responsive medium generates acoustic waves (e.g., 380) at about the target frequency. For example if the field oscillating device includes a coil 370, oscillation of a magnetic field can be accomplished via an oscillating current source 340 with optional additional circuitry 350. An optional shield 330 (e.g., EM, mu metal, microwave) can encompass the field oscillating device to avoid any harmful waves reaching a user or patient.

In the alternative an impinging pressure wave will move the field responsive medium 320 within a background field generated by the field oscillating device generating a current that oscillates in response to the movement of the field responsive medium 320. Thus the system can additionally or alternatively act as a microphone.

FIG. 4 illustrates at least one exemplary embodiment of generating acoustic waves using an oscillating field responsive medium (e.g., an electro responsive fluid (ER)). In the non-limiting example illustrated the field responsive medium 420 responds to electric fields generated by a varying potential difference between a first electrode 320A and a second electrode 320B. The varying potential can be generated by an oscillating voltage source 410 is coupled to the first electrode 320A and the second electrode 320B with optional additional circuitry 350. As the voltage is changes the field responsive medium moves, generating acoustic waves 380. Alternatively impinging pressure waves can oscillate the field responsive medium 420, varying the capacitance between the first (320A) and second (320B) electrodes which can, as also discussed with respect to FIG. 3, be converted into electronic signals representing the pressure waves. Thus at least one exemplary embodiment using an electric field responsive medium and/or magnetic field responsive medium, can be used as a microphone. Note that microphone is a general term not intended to limit the frequency of the acoustic or pressure waves detected. Note also a cooling coil and/or radiator 710 can be used to cool the system (e.g. the field generating device) of the field responsive medium systems (e.g., FIG. 3 and FIG. 4), in addition to the laser systems described (e.g., FIG. 5, FIG. 6, and FIG. 7).

FIG. 5 illustrates at least one exemplary embodiment 500 of generating acoustic waves using photo acoustics. An acoustic wave can also be generated by a pulsed laser generating a laser pulse 510, travelling through a first medium 540 (e.g., air), that impinges upon a laser reactive material 530, that oscillates in response to the laser pulse 510. For example a material that expands (e.g., thermal expansion) and contracts at a frequency corresponding to the pulse frequency. The expansion and contraction generates an acoustic wave 580. The laser can optionally be delivered to the laser reactive material 530 via a fiber optic cable 520. Note that in the alternative an impinging pressure field on a layer (e.g., the laser reactive material 530, or in this case even a material that reflects at least a portion of the incident laser pulse) will oscillate the layer. The oscillation of the layer can be detected by ranging the laser pulses (e.g., detecting a Doppler shift) thus acting as a laser reflective based microphone. The microphone can be as small as a fiber optic cable where the layer can oscillate in response to a pressure wave.

FIG. 6 illustrates at least one exemplary embodiment 600 of generating acoustic waves using photo acoustics in one medium 540 and coupling the generated acoustic waves into a second medium 630. An acoustic bridging medium 610 can be used to isolate the first medium 540 (e.g., air) from the second medium 630 (e.g., water) and still transmit (620) at least a portion of the generated 580 or received acoustic wave between the two mediums (540 and 630).

FIG. 7 illustrates at least one exemplary embodiment of generating acoustic waves, with optional cooling coils and/or radiators 710 and optional shielding 330. In some cases the frequencies generated may heat materials and the materials may need to be cooled. Additionally certain frequencies can generate EM wavelengths that might be harmful (e.g., burning) and thus shielding can be used.

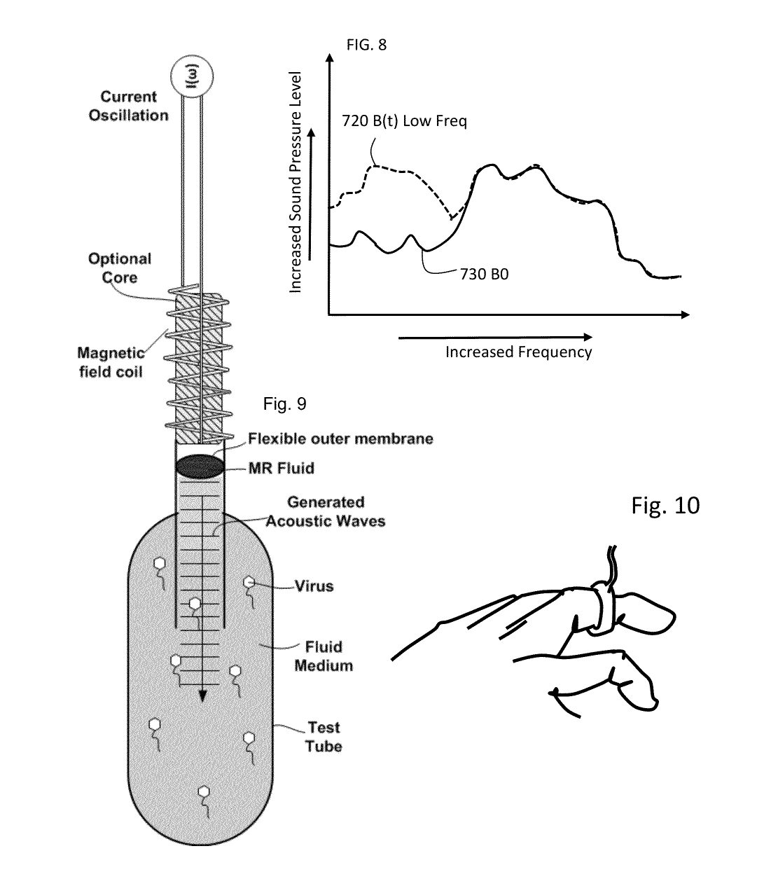

FIG. 8 illustrates data from a proof of concept experiment generating an acoustic wave in from an oscillating MR fluid bladder by changing the magnetic field at low frequencies. A background acoustic field 830 is measured with a stable magnetic field B0. A field oscillating device began a low frequency broadband oscillation stimulating oscillation of a responsive field medium which generated the acoustic waves 820 at the varying magnetic field frequencies B(t). In the limited example the increase in sound pressure level was about 30 dB.

FIG. 9 illustrates an example of using an acoustic generated acoustic wave 380 to sterilize a viral 220 infected medium 910 in a test tube 900.

FIG. 10 illustrates at least one exemplary embodiment 1010 of coupling an acoustic wave (e.g. via acoustic waveguide) 1000 to a ring device 1030 on a finger 1020 to input the acoustic waves into a patient's body. Note that eradication of the entire targeted virus is not necessary in some cases where the decreased level of live virus can provide the body's immune system to eradicate or control the remaining level of viruses.

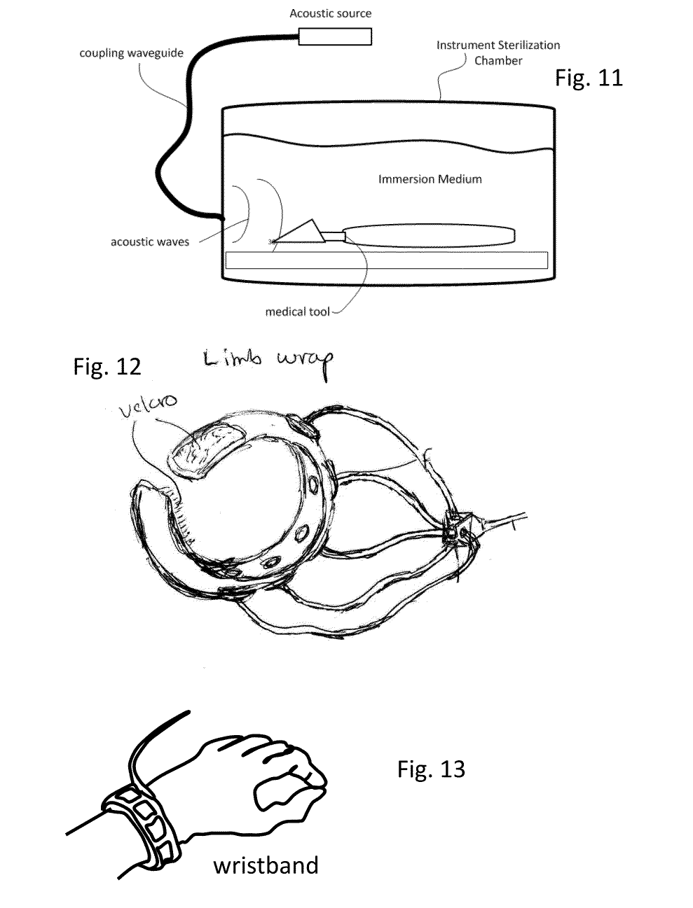

FIG. 11 illustrates at least one exemplary embodiment 1100 of coupling an acoustic wave (e.g. via acoustic waveguide) to an instrument 1160 (e.g., medical surgical instrument) to sterilize (eradicate a portion of the population of the targeted virus attached to) the instrument 1160. In this non limiting example the acoustic source 1110 generates an acoustic wave that is coupled 1130 (e.g. waveguide) to an instrument sterilization chamber 1120. The acoustic waves are tuned for a targeted viruses resonance frequency. The acoustic waves traveled through an immersion medium (e.g., water) 1150 to the instrument 1160 (e.g., surgical device), setting up a vibration in the viruses that eradicate a certain number of the viruses within and exposure time. Note alternatively the instrument 1160 itself can be vibrated (e.g., via phonons or a mechanical vibrator) at the viral target frequency to destroy targeted viruses. In such a system the acoustic source can be a vibration system coupled to the instrument 1160.

FIG. 12 illustrates at least one exemplary embodiment of coupling an acoustic wave (e.g. via acoustic waveguide) to a limb wrap device 1200, that can be wrapped around a limb to input the acoustic waves into the patient body. For example an acoustic source can be coupled via a waveguide 1210 that can be coupled to multiple waveguides 1220, where the multiple waveguides deliver an acoustic signal to various locations 1250 that touch the patient's skin. The various locations can be part of a wrap 1240 that can be secured around a limb using a fastening method 1230 (e.g. Velcro™).

FIG. 13 illustrates at least one exemplary embodiment 1300 of coupling 1320 an acoustic wave (e.g. via acoustic waveguide) to a wrist wrap device 1330, that can be wrapped around a wrist 1310 to input the acoustic waves into the patient body.

As discussed above one can vary current and/or voltage to generate acoustical energy. Note also that if a steady field is imposed, then when sound impinges upon a field responsive medium (liquid, gas, solid) an induced current and/or voltage is generated. The induced current and/or voltage can be converted by known methods to pick up sound thus the systems described can also in certain configurations act as microphones.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions of the relevant exemplary embodiments. For example, if words such as “orthogonal”, “perpendicular” are used the intended meaning is “substantially orthogonal” and “substantially perpendicular” respectively. Additionally although specific numbers may be quoted in the claims, it is intended that a number close to the one stated is also within the intended scope, i.e. any stated number (e.g., 20 mils) should be interpreted to be “about” the value of the stated number (e.g., about 20 mils).

Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

What is claimed is:

1. An acoustic generation device comprising:
a field oscillating device; and
a field responsive medium, where the field oscillating device is configured to generate an oscillating field at a target frequency moving the field responsive medium, where movement of the field responsive medium generates acoustic waves at about the target frequency.

2. An acoustic receiving device comprising:
a field oscillating device; and
a field responsive medium, where the field oscillating device is configured to generate an oscillating current at a target frequency when the field responsive medium oscillates in response to an impinging pressure wave at the target frequency, where the oscillating current is generated by changing fields in the field oscillating device as the field responsive fluid oscillates.

3. A method of viral eradication comprising:
delivering an acoustic wave to a virally infected region;
tuning a frequency of the acoustic wave to a resonance frequency of a target virus in the virally infected region; and
applying the acoustic wave to the virally infected region for a period of time necessary to eradicate at least 25% of the target virus per cubic mm of the virally infected region.
